(19) United States
(12) Patent Application Publication
(10) Pub. No.: US 2019/0262158 A1
(43) Pub. Date: Aug. 29, 2019

(54) GASTRIC BYPASS DEVICE

(71) Applicant: Cook Medical Technologies LLC, Bloomington, IN (US)

(72) Inventors: Kalub Hahne, West Lafayette, IN (US); Barrett Davis, Lafayette, IN (US)

(73) Assignee: Cook Medical Technologies LLC, Bloomington, IN (US)

(21) Appl. No.: 16/281,780

(22) Filed: Feb. 21, 2019

Related U.S. Application Data
(60) Provisional application No. 62/634,454, filed on Feb. 23, 2018.

(51) Int. Cl. A61F 5/00; A61F 2/04

(52) U.S. Cl. CPC A61F 5/0076; A61F 5/0036; A61F 2/04; A61F 2250/0039; A61F 2002/045; A61F 2210/0057; A61F 2230/0013; A61F 2002/044

(57) ABSTRACT

A gastric bypass device may include a first portion with an opening sized for receiving GI contents within an esophagus of the gastrointestinal tract, where a proximal end of the first portion includes first diameter, and where the first portion includes a first stent for anchoring the proximal end of the first portion. The gastric bypass device may also include a second portion located distally of the first portion, where the second portion is in fluid communication with the first portion, and where the second portion has an enlarged section with a second diameter that is at least about 150% as large as the first diameter.

### RELATED APPLICATIONS

This application claims the benefit of U.S. Provisional Application No. 62/634,454, filed Feb. 23, 2018, which is hereby incorporated by reference in its entirety.

### BACKGROUND

Non-alcoholic steatohepatitis (NASH) is a disease subset of nonalcoholic fatty liver disease (NAFLD) that the National Institutes of Health (NIH) characterizes by histologic evidence of liver steatosis, lobular inflammation and hepatocellular ballooning. NASH is a known contributor to liver cancer and cirrhosis, and if left untreated, NASH will eventually lead to end-stage liver disease. Because NASH is largely considered a “silent” disease (portraying few or no outward physical symptoms), there is a paucity of literature on its incidence rate. However, the internal damage it causes is clear and, NASH is predicted by some to become the leading indication for liver transplantation in the next ten to twenty years. NASH primarily affects middle-aged, overweight, and obese individuals, and in the United States, it has been reported in as many as 37% of patients who undergo bariatric surgery (commonly indicated by a BMI of at least 40%), 18.5% of otherwise-markedly obese individuals, and 2.7% of lean individuals. Overall, an estimated 30% of Americans—roughly 110 million people—are said to be effected by NASH. With a 42% predicted prevalence of obesity in American adults by the year 2030, this number—and the share of those affected by NASH within it—will likely only increase with time.

Currently, the most effective way of treating NASH is bariatric surgery, where following the surgical intervention, NASH has been reported to improve in 60-80% of cases. However, bariatric surgery is not without drawbacks. The most popular gastric bypass methods (e.g. Roux-en-Y, sleeve gastrectomy, and adjustable gastric banding) are very invasive and, even if done laparoscopically, pose significant challenges due to the prevalence of post-operative infections (approximately 15% of all patients contract surgical site infections), lengthy recoveries (2-3 days minimum), and delayed starts to active treatment (3-6 weeks). In addition, only the Roux-en-Y and sleeve gastrectomy—the two most invasive of the three most common bariatric procedures—have been demonstrated to reduce the long-term blood concentration of the hunger hormone acyl-Ghrelin more so than control groups that lost similar amounts of weight by non-surgical means. Although the exact action of acyl-Ghrelin is not yet fully understood, it is believed that gastric Ghrelin production is highly dependent on the contact between food and gastric cells.

In view of this background, it would be advantageous to provide a less-invasive gastric bypass solution that limits the interaction of food with the stomach and can provide a smaller risk of infection, a shorter recover time, and more immediate start to treatment.

### BRIEF SUMMARY

In one aspect, a gastric bypass device may include a first portion with an opening sized for receiving GI contents within an esophagus of the gastrointestinal tract, where a proximal end of the first portion includes first diameter, and where the first portion includes a first stent for anchoring the proximal end of the first portion. The gastric bypass device may also include a second portion located distally of the first portion, where the second portion is in fluid communication with the first portion, and where the second portion has an enlarged section with a second diameter that is at least about 150% as large as the first diameter.

In some embodiments, the gastric bypass device includes a third portion located distally of the second portion, where the third portion includes a third diameter that is smaller than the second diameter. A distal end of the third portion may include a distal-end stent for anchoring the distal end of the third portion in a duodenum of the gastrointestinal tract. The third portion may include a compliant area located proximally of the distal-end stent. A distal end of the third portion may include a distal-end diameter that is at least about 20% larger than the third diameter of the third portion. The third portion may have a tendency to curve when deployed.

In some embodiments, the enlarged section of the second portion includes a second stent that provides the second diameter. The enlarged section of the second portion may have a stretchable cover for forming a pocket upon receipt of GI contents. A covering surrounding the enlarged section of the second portion may be at least partially permeable by certain liquids.

### BRIEF DESCRIPTION OF THE DRAWINGS

FIG. 1 is an illustration showing a gastric bypass device deployed within a human patient in accordance with certain embodiments of the present disclosure.

FIG. 2 is an illustration showing the gastric bypass device of FIG. 1 in isolation.

FIG. 3 is an illustration showing a gastric bypass device having an enlarged section formed by a plurality of stents in accordance with certain embodiments of the present disclosure.

### DETAILED DESCRIPTION

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The invention is described with reference to the drawings in which like elements are referred to by like numerals. The relationship and functioning of the various elements of this invention are better understood by the following detailed description. However, the embodiments of this invention are not limited to the embodiments illustrated in the drawings. It should be understood that the drawings are not to scale, and in certain instances details have been omitted which are not necessary for an understanding of the present invention, such as conventional fabrication and assembly.

As used in the specification, the terms proximal and distal should be understood as being in the terms of a physician delivering the medical device to a patient. Hence the term “distal” means the portion of the medical device that is farthest from the physician and the term “proximal” means the portion of the medical device that is nearest to the physician.

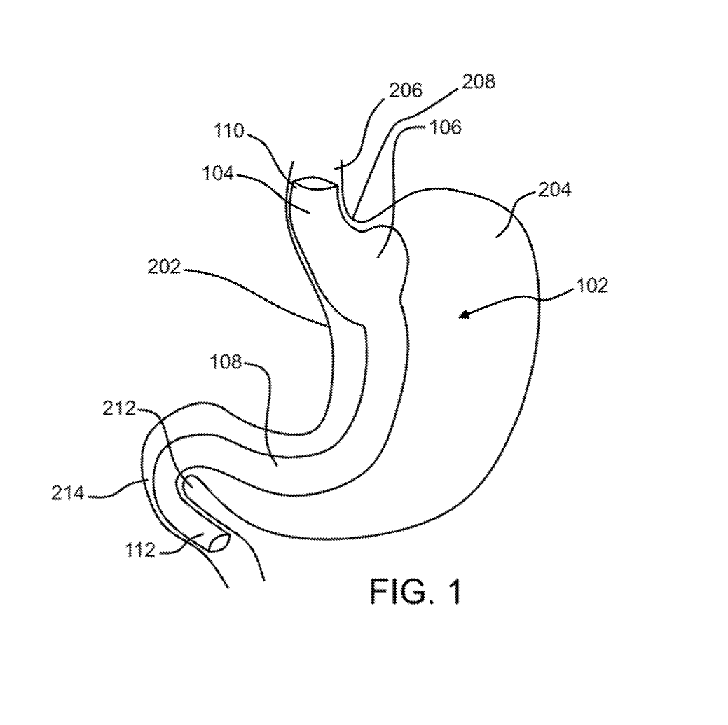

FIG. 1 is an illustration showing a gastric bypass device 102 deployed within a gastrointestinal tract 202 of a human. Providing the gastric bypass device 102 may provide the benefits of other gastric bypass procedures by directing GI contents (i.e., food and other ingested matter passing through the gastrointestinal tract along) such that they bypass at least a portion of the stomach 204, thus reducing the functional size of the gastrointestinal tract 202 to limit the amount of food a patient may comfortably ingest. Further, since the gastric bypass device 102 may include a covering to prevent food from exposure to gastric cells (as described in more detail below), the long-term exposure of gastric cells to boluses of food may be advantageously reduced.

In the depicted embodiment, the gastric bypass device 102 may include a first portion 104 at a proximal end 110 of the gastric bypass device, a second portion 106 extending distally from the first portion 104, and a third portion 108 extending distally from the second portion 106 to a distal end 112. When the gastric bypass device 102 is deployed, the first portion 104 may be anchored to a lower (distal) portion of the esophagus 206. The second portion 106 may extend distally from the first portion 104 and through at least a portion of the stomach 204 (starting approximately from an esophageal sphincter 208). The third portion 108 may extend distally from the second portion 106 and serve as an outlet to the second portion 106. The third portion 108 may further extend through the pyloric sphincter 212 and then terminate at the distal end 112 at a location within the upper intestinal tract or duodenum 214.

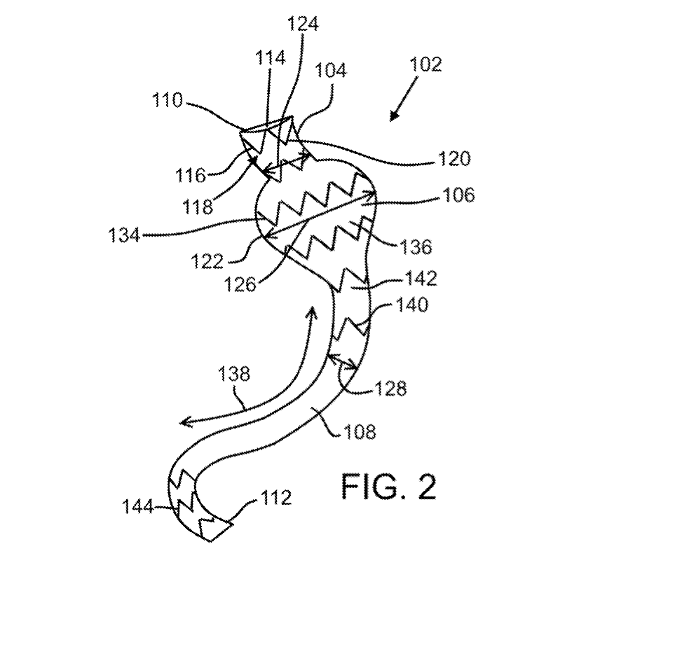

FIG. 2 is an illustration showing the gastric bypass device 102 of FIG. 1 in isolation. The first portion 104 may include the proximal end 110 for deployment within the esophagus 206 (e.g., above/proximal of the esophageal sphincter). The first portion 104 may have a mouth 114 for receiving food or other GI contents as they move distally through the gastrointestinal tract. In some embodiments, the first portion 104 may include a first stent 116, which may have an outer surface 118 for engaging the inner wall of the esophagus 206 (see FIG. 1) to anchor the first portion 104 in place. The first stent 116 may be expandable during deployment, and may be self-expanding or may expand under pressures or other influence from another device, such as an inflatable balloon at the tip of a balloon catheter. The first stent 116 may include any suitable stent pattern. One example of a stent pattern is the Z-stent or Gianturco stent design (Cook® Medical). The stent pattern may include a series of substantially straight segments or struts 120 interconnected by a series of bent segments or bends. The bent segments may include acute bends or apices. The segments may be arranged in a zigzag configuration where the straight segments are set at angles relative to one another and are connected by the bent segments. The first stent 116 may additionally or alternatively be formed of another stent pattern, such as an annular or helical stent pattern. Without limitation, the segments mentioned herein may be made from standard medical grade stainless steel or from nitinol or other shape-memory materials, and/or other metals/polymers in either a permanent or bioabsorbable design. The first stent 116 may have features similar or identical to an embodiment of an Evolution® Esophageal Controlled-Release Stent sold by Cook® Medical. It is also contemplated that a different support structure may be used with the first portion 104 instead of (or in addition to) a stent, such as a substantially rigid or obstinate tube formed of a biocompatible material, of any other suitable device for providing support and/or preventing migration.

As shown in FIG. 2, the second portion 106 of the gastric bypass device 102 may extend distally from the first portion 104. The second portion 106 may be in fluid communication with the first portion 104 such that GI contents passing distally through the first portion 104 are received by the second portion 106. As shown, the second portion 106 may include an enlarged section 122, which may function in a manner similar to a stomach pouch used in Roux-en-Y procedures and may partly be supported by the upper walls of the stomach 204. The enlarged section may, for example, allow foods and other GI contents to partially digest before moving beyond the stomach even such that the GI contents do not need to be highly liquified within the enlarged section 122. As it extends distally from approximately the esophageal sphincter 208, the second portion 106 may quickly increase from a first diameter 124 adjacent to the beginning of the enlarged section 122 to a second diameter 126 extending around the largest cross-section of the enlarged section 122. While any suitable diameters are contemplated, in certain non-limiting exemplary embodiments, the first diameter 124 of the first portion 104 may be between about 5 mm and about 40 mm, such as between about 15 mm and about 25 mm, and such as about 20 mm when used in a normal-sized adult human. In some embodiments, the cross-section of the first portion 104 may be ovular, or may include another shape. Thus, the first diameter 124 (or other cross-sectional dimension) may be suitable for engagement with inner walls of the esophagus 206.

The second diameter 126, which may be a maximum diameter of the second portion 106, may be between about 30 mm and about 60 mm, such as from about 34 mm to about 45 mm in non-limiting exemplary embodiments. Thus, the second diameter 126 may be about 150% to about 300% of the first diameter 124, such as from about 170% to about 225%. Other dimensions and shapes are also contemplated.

The second portion 106 may include any suitable construction. For example, in some embodiments, the second portion 106 may be formed by a covering with extra material (e.g., polytetrafluoroethylene (PTFE)) forming a pocket (and it is noted that the “second diameter 126” may be a diameter when the pocket is in a filled or otherwise expanded state). Additionally or alternatively, the material forming the second portion 106 may include a stretchable or expandable material such that the second portion 106 has different dimensions depending on whether or not it includes GI contents. Optionally, the second portion 106 may include at least one second stent 134, which may include any of the features with respect to the first stent 116 described above (e.g., it may be expandable during deployment of the gastric bypass device 102). In some embodiments, the stent 134 may be formed of a reinforced polymer material and/or PTFE.

Preferably, food and/or other GI contents are substantially prevented from escaping through the walls of the second portion 106 and into bypassed portions of the gastrointestinal tract (though it is contemplated that, in some embodiments, the walls of the second portion 106 may be configured to allow certain liquids or other fluids to permeate through). When the second stents 134 are included, they may be covered stents, and thus may include a covering 136 sealing the walls of the second portion 106. The covering may be formed of any suitable material, such as a polytetrafluoroethylene (PTFE). The covering 136 could be permeable, partially permeable, or impermeable to fluids. In some embodiments, the covering is a permeable woven or ePTFE which allows for the movement of fluids from inside to outside the second portion 106, and vice versa. The covering 136 (or other sealing device) may serve a variety of functions. For example, the covering 136 may retain most or all of the GI contents (particularly solid contents) within the gastric bypass device 102 such that they do not leak into bypassed portions of the stomach 204. Additionally (or alternatively), the covering 136 may prevent interactions between food or other GI contents and the gastric cells, which may therefore reduce gastric Ghrelin production.

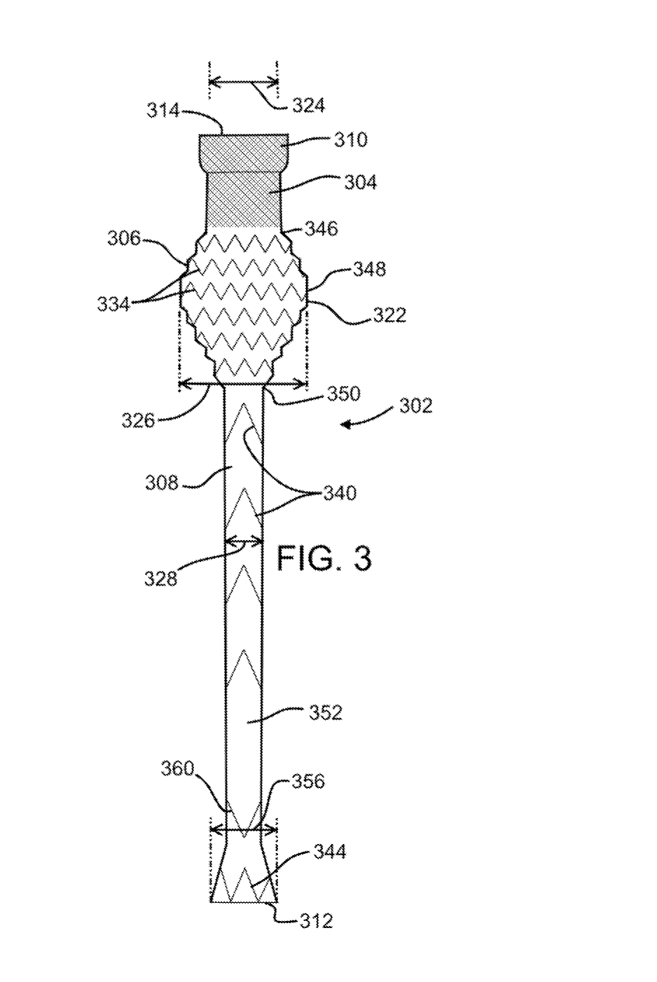

The third portion 108 may extend distally from the second portion 106, thus providing an outlet from the enlarged section 122 of the gastric bypass device 102. The third portion 108 may include any suitable construction (e.g., a flexible tube, a stented expandable tube, etc.). In some embodiments (without limitation), the third portion 108 may be constructed from a length of flexible braided nitinol, with nitinol ring supports or ring supports of another material, and/or a polymeric stent structure. These construction options may be advantageous for reducing potential gastric irritation from stent contact with the pyloric sphincter and its aggressive peristalsis. Thus, the third portion 108 may include a length that lacks stent material (e.g., metal, such as nitinol, or another suitable material) where the third portion 108 will interact with the pyloric sphincter. Particularly when the covering of the third portion 108 is flexible, an area free of a stent may be compliant such that it allows the pyloric sphincter to close/open the passage through the third portion 108 to control digestion (see, e.g., the compliant area 352 described with reference to FIG. 3).

In some embodiments, the third portion 108 may, as depicted, extend at an angle and/or with curvature to accommodate the asymmetric anatomy of the stomach 204. The angle and/or curvature may be provided by the construction of the third portion 108 such that the third portion 108 has a natural tendency to curve (e.g., along the depicted curvature 138) with respect to its longitudinal direction when straightened. For example, when the third portion 108 includes one or more optional third stents 140, the third stents 140 may be formed with a material (e.g., nitinol) and/or with a particular strut arrangement such that the third portion 108 includes the tendency to curve. The inherent curvature may be advantageous for assisting in proper positioning of the gastric bypass device 102 during its deployment. However, in other embodiments, the third portion 108 may be flexible such that its curvature is simply an effect of the positioning of its proximal end 142 at the enlarged section 122 and its distal end 112 at the pyloric sphincter 212 (as discussed below with reference to FIG. 3).

The distal end of the third portion 108 may extend through the pyloric sphincter and into the duodenum when the gastric bypass device 102 is deployed (as shown in FIG. 1). To anchor the distal end 112 of the third portion 108 within the duodenum, the third portion 108 may include a fourth stent 144 on its distal end. The fourth stent 144 may be an expandable stent an may incorporate any features with respect to the stents described above (and/or any other suitable feature). While any deployment order is contemplated, the fourth stent 144 may be delivered first to anchor the third portion 108. Then, other stents located proximally may be deployed to anchor the respective portions of the gastric bypass device 102 in their respective locations.

The third portion 108 may include a third diameter 128, which may be smaller than at least the second diameter 126, and in some embodiments the third diameter 128 may also be smaller than the first diameter 124. In certain non-limiting exemplary embodiments for a normal-sized adult, the third diameter 128 may be about 11 mm. This diameter may be appropriate for anchoring the third portion 108 within the duodenum in a normal sized adult. However, other diameters are contemplated, and the diameter of the third portion 108 may vary along its length (e.g., as shown in FIG. 3).

In some embodiments, the distal end 112 of the third portion 108 may expand in diameter. For example, a diameter (or other cross-sectional dimension) at the distal end 112 may be about 10% larger to about 100% larger, such as about 20% larger, than a diameter of the third portion 108 positioned near/within the pyloric sphincter. The expansion may be accomplished by way of the stent 144 located at the distal end 112. The expanded diameter (or other cross-sectional dimension) of the distal end 112 may be advantageous for anchoring the distal end 112 within the duodenum. For example, the distal end 112 may press against the or otherwise engage the inner-facing surfaces of the duodenum, and or the distal end 112 may be sized such that it is too large to be pulled proximally through the pyloric sphincter. While not shown, the distal end 112 may include barbs or other securement devices to enhance anchoring.

The gastric bypass device 102 includes several advantageous features. For example, the gastric bypass device 102 provides an alternative bariatric method for treating NASH that has fewer costs, a shorter recovery period, a near immediate start to active treatment and better long-term patient outcomes. Further, the gastric bypass device 102 may reduce follow-up appointments and potential infection-treatment. As for better long-term patient outcomes, in addition to mitigated risks of surgical site infections, patients treated by the gastric bypass device 102 would benefit from a long-term reduction in acyl-Ghrelin production from the covered sections of the stent preventing contact between food and gastric cells.

Another embodiment of a gastric bypass 302 device is shown in FIG. 3. As shown, a first portion 304 includes a first diameter 324 for cooperation with the inner walls of an esophagus, and a proximal end 310 of the first portion 304 has a mouth 314 (e.g., an opening) for receiving GI contents as they move distally through the esophagus. While not required, the proximal end 310 is shown as having a slightly larger diameter than the remainder of the first portion 304, which may be advantageous for ensuring the proximal-most location of the first portion 304 is sealingly engaged with the esophagus wall such that GI contents cannot bypass the mouth 314. As shown, the outer surface of the first portion 304 may include a bioabsorbable material such that is absorbed by the esophageal tissue over time.

A second portion 306 may include an enlarged section 322 with a second diameter 326, which may be advantageous for the reasons described with respect to the above embodiments. To ensure that the gradient (e.g., slope of the change in cross section) is gradual (e.g., to avoid “dead spots” where GI contents get stuck), the second portion 306 may include a series of second stents 334. Thus, the stents may increase in relative size from the proximal end 346 of the second portion 306 to the location 348 of maximum enlargement, and then decrease in relative size from the location 348 to the distal end 350 of the second portion 306. Any number of stents are contemplated (such as 6, as shown in FIG. 3).

The third portion 308 (with a third diameter 328) may include one or more third stents 340 to ensure structural integrity of the third portion 308. A compliant area 352 may correspond with the pyloric sphincter such that the pyloric sphincter can continue to operate (e.g., to interrupt fluid communication with the distal end) to properly control GI-content flow. The compliant area 352 may have a natural tendency to assume a tubular shape (e.g., such that it opens when the pyloric sphincter opens), but it may be compliant enough as to not interrupt operation of the pyloric sphincter. In some embodiments, the compliant area 352 is a region without a stent (or, a compliant stent) located between the most proximal stent of the third stents 340 and a distal-end stent (e.g., a fourth stent 344). In some embodiments, an optional fifth stent 360, which may be similar or identical to the third stents 340, may be configured for deployment distally of the compliant area and located proximally of the fourth stent 244.

The distal end 312 of the third portion 308, which may be located distally of the pyloric sphincter and in the duodenum when deployed, may include the fourth stent 344, which may form a fourth diameter 356 larger than the third diameter 328. As described in more detail above (with reference to distal end 112 of FIG. 2), the fourth diameter 356 may be sized such that the distal end 312 of the third portion 308 cannot migrate proximally through the pyloric sphincter.

The above figures and disclosure are intended to be illustrative and not exhaustive. This description will suggest many variations and alternatives to one of ordinary skill in the art. All such variations and alternatives are intended to be encompassed within the scope of the attached claims. Those familiar with the art may recognize other equivalents to the specific embodiments described herein which equivalents are also intended to be encompassed by the attached claims.

We claim:

1. A gastric bypass device for use in a gastrointestinal tract, comprising:
a first portion with an opening sized for receiving GI contents within an esophagus of the gastrointestinal tract, wherein a proximal end of the first portion includes a first diameter, and wherein the first portion includes a first stent for anchoring the proximal end of the first portion; and
a second portion located distally of the first portion, wherein the second portion is in fluid communication with the first portion, and wherein the second portion has an enlarged section with a second diameter that is at least about 150% as large as the first diameter.

2. The gastric bypass device of claim 1, further comprising:
a third portion located distally of the second portion,
wherein the third portion includes a third diameter that is smaller than the second diameter.

3. The gastric bypass device of claim 2, wherein a distal end of the third portion includes a distal-end stent for anchoring the distal end of the third portion in a duodenum of the gastrointestinal tract.

4. The gastric bypass device of claim 3, wherein the third portion includes a compliant area located proximally of the distal-end stent.

5. The gastric bypass device of claim 2, wherein a distal end of the third portion includes a distal-end diameter that is at least about 20% larger than the third diameter of the third portion.

6. The gastric bypass device of claim 2, wherein the third portion includes a tendency to curve when deployed.

7. The gastric bypass device of claim 1, wherein the enlarged section of the second portion includes a second stent that provides the second diameter.

8. The gastric bypass device of claim 1, wherein the enlarged section of the second portion includes a stretchable cover for forming a pocket upon receipt of GI contents.

9. The gastric bypass device of claim 1, further comprising a covering surrounding the enlarged section of the second portion, the covering being at least partially permeable by certain liquids.

10. A gastric bypass device for use in a gastrointestinal tract, comprising:
a first portion having a first diameter;
a second portion located distally of the first portion, the second portion having a second diameter that is larger than the first diameter; and
a third portion located distally of the second portion, wherein the third portion includes a third diameter that is smaller than the second diameter, and wherein a distal end of the third portion includes a distal-end stent for anchoring the distal end of the third portion.

11. The gastric bypass device of claim 10, wherein the second diameter is at least about 150% as large as the first diameter.

12. The gastric bypass device of claim 10, further comparing a stent located at a proximal end of the third portion and adjacent to the second portion.

13. The gastric bypass device of claim 12, wherein the third portion includes a compliant area located between the stent and the distal-end stent.

14. The gastric bypass device of claim 10, wherein the distal end of the third portion includes a distal-end diameter that is at least about 20% larger than the third diameter of the third portion.

15. The gastric bypass device of claim 10, wherein the third portion includes a tendency to curve when deployed.

16. The gastric bypass device of claim 10, wherein the second portion includes a second stent that provides the second diameter.

17. The gastric bypass device of claim 10, wherein the second portion includes a stretchable cover for forming a pocket upon receipt of GI contents.

18. A method, comprising:
deploying a first portion of a gastric bypass device within a gastrointestinal tract, the first portion having an opening sized for receiving GI contents within an esophagus of the gastrointestinal tract, wherein a proximal end of the first portion includes a first diameter, and wherein the first portion includes a first stent for anchoring the proximal end of the first portion; and
deploying a second portion of the gastric bypass device such that the second portion is located distally of the first portion, wherein the second portion is in fluid communication with the first portion, and wherein the second portion has an enlarged section with a second diameter that is at least about 150% as large as the first diameter.

19. The method of claim 18, further comprising:
deploying a third portion of the gastric bypass device such that the third portion is located distally of the second portion,
wherein the third portion includes a third diameter that is smaller than the second diameter.

20. The method of claim 19, wherein a distal end of the third portion includes a distal-end stent for anchoring the distal end of the third portion in a duodenum of the gastrointestinal tract.
